(19) United States
(12) Patent Application Publication
(10) Pub. No.: US 2024/0216698 A1
(43) Pub. Date: Jul. 4, 2024

(54) METHOD AND SYSTEM FOR IMPLANTING A SEPTAL WALL ELECTRODE

(71) Applicant: Pacesetter, Inc., Sylmar, CA (US)

(72) Inventors: Jan O. Mangual-Soto, Rho (IT); Nikolaos Politis, Berlin (DE)

(21) Appl. No.: 18/497,593

(22) Filed: Oct. 30, 2023

Related U.S. Application Data
(60) Provisional application No. 63/477,509, filed on Dec. 28, 2022.

(51) Int. Cl. A61N 1/37; A61N 1/05

(52) U.S. Cl. CPC A61N 1/3702; A61N 1/057; A61N 2001/058

(57) ABSTRACT

A system is provided that includes a lead configured to be located within a septal wall, a monitor configured to obtain cardiac activity signals, and a memory configured to store program instructions. The system also includes one or more processors that, when executing the program instructions, are configured to obtain morphology data related to the cardiac activity signals indicative of the lead located at different depths within the septal wall, the morphology data including a set of data values associated with different depths of the lead within the septal wall, and determine when the lead is located at a target depth within the septal wall based on the morphology data.

### CROSS REFERENCE

This Application claims priority to U.S. Provisional Application No. 63/477,509 filed 28 Dec. 2022 entitled METHOD AND SYSTEM FOR IMPLANTING A SEPTAL WALL ELECTRODE, the entire disclosure of which is incorporated herein by reference.

### FIELD OF THE INVENTION

Embodiments herein relate generally to implantable medical devices, and more particularly to the advancing of an electrode through a septal wall during implant.

### BACKGROUND OF THE INVENTION

For some time right ventricular (RV) pacing has been utilized in patients with atrioventricular (AV) block. However, in certain instances, RV pacing has been shown to induce electrical and mechanical dyssynchrony. HIS bundle pacing (HBP) has been proposed as an alternative to RV pacing, taking advantage of physiological conduction, resulting in synchronous ventricle contraction. HBP has also been proposed as an alternative to biventricular cardiac resynchronization therapy (CRT) in patients with left bundle branch block (LBBB). Nevertheless, HBP still experiences challenges in identifying and targeting the HIS bundle, lead stability, and long-term pacing threshold.

A technique has been developed to directly capture left bundle branch (LBB) conduction by going deep into the ventricular septum. In particular, LBB pacing (LBBp) is clinically feasible in heart failure (HF) patients with LBBB and indication for cardiac resynchronization therapy (CRT). In particular, LBBp can potentially overcome limitations in HBP and offer a safer technique for conduction system pacing.

The location of the LBB, and thus target depth of an electrode at the distal tip of the lead is generally 1 to 1.5 cm distal along the septal wall of the HIS. During lead placement, the pacing lead is placed, or screwed into the myocardium on the right ventricle side and tunneled into the septum. This procedure is provided by a surgeon who has a limited ability to determine in real-time when the lead is located on the RV septum side wall, the middle on the intraventricular septum (e.g. mid-septum wall), and on the LV septum side wall. Placement of the lead such that the electrode at the distal tip of the lead is proximate the RBB in the septal wall is undesired and represents an unsuccessful implant. Additionally, advancing the lead too far through the LV septum side wall resulting in perforating the LV septum side wall into a left chamber of the heart is similarly undesired and problematic. Because the target depth proximate the LBB has a small range, greater accuracy in placement is desired.

### SUMMARY

In accordance with embodiments herein, a system is provided that includes a lead configured to be located within a septal wall, a monitor configured to obtain cardiac activity signals, and a memory configured to store program instructions. The system also includes one or more processors that, when executing the program instructions, are configured to obtain morphology data related to the cardiac activity signals indicative of the lead located at different depths within the septal wall, the morphology data including a set of data values associated with different depths of the lead within the septal wall, and determine when the lead is located at a target depth within the septal wall based on the morphology data.

Optionally, the target depth locates the lead proximate to the left bundle branch (LBB). In one aspect, the morphology data includes QRS width of consecutive heartbeats, and the one or more processors are configured to determine that the lead is located proximate the LBB based on a change in the QRS width from a first QRS width to a second QRS width. In another aspect, the change in the QRS width is greater than a QRS width threshold change between the first QRS width and the second QRS width. Optionally, the first QRS width is obtained from a first heartbeat and the second QRS width is obtained from a second heartbeat, and the first heartbeat and second heartbeat are not consecutive heartbeats. In one example, the morphology data includes a peak-to-peak amplitude of consecutive heartbeats, and the one or more processors are configured to determine that the lead is located proximate the LBB based on a change in a peak-to-peak amplitude value between a first peak-to-peak amplitude and a second peak-to-peak amplitude. In another example, the change in peak-to-peak amplitude is greater than a threshold change between the first peak-to-peak amplitude and the second peak-to-peak amplitude. In yet another example, wherein the determine operation includes determining when the data values satisfy a criteria of interest. Alternatively, the determine operation further comprises comparing the data values, within the set of data values, to one another to determine a relation; and determining when the relation satisfies the criteria of interest. Additionally, the criteria of interest can be at least one of change in QRS width, or a change in a peak-to-peak amplitude. Further, the target depth includes a point where the lead is proximate the LBB and does not perforate a LV septum side wall distal wall of the septal wall. Optionally, the lead is a first electrode of a leadless IMD.

In accordance with embodiments herein, a computer implemented method is provided, where under control of one or more processors configured with specific executable instructions, the method includes obtaining morphology data indicative of a morphology of consecutive beats of a heart including a set of data values associated with different depths of a lead within the septal wall. The method also includes determining when the lead is located at a target depth within the septal wall based on the morphology data.

Optionally, the method also includes determining that the lead is located proximate the LBB when the morphology data indicates a change in QRS width is above a QRS width change threshold. In one aspect, the method additionally includes determining that the lead is located proximate the LBB when the morphology data indicates a change in peak-to-peak amplitude is above a peak-to-peak amplitude change threshold. In another aspect, the determining operation includes determining when the data values satisfy a criteria of interest. In one example, the determining operation further comprises comparing the data values, within the set of data values, to one another to determine a relation; and determining when the relation satisfies the criteria of interest. In another example, the criteria of interest is at least one of change in QRS width, or a change in a peak-to-peak amplitude. In yet another example, the target depth includes a point where the lead is proximate the LBB and does not perforate a LV septum side wall distal wall of the septal wall. Optionally, obtaining morphology data indicative of the morphology of the consecutive beats of the heart comprises monitoring, with a monitor, the consecutive beats of the heart as the lead is inserted into the septal wall.

### BRIEF DESCRIPTION OF THE DRAWINGS

FIG. 1 illustrates an implantable medical device (IMD) intended for subcutaneous implantation at a site near the heart in accordance with embodiments herein.

FIG. 2 illustrates a block diagram of an IMD in accordance with embodiments herein.

FIG. 3 illustrates an IMD intended for implantation in the heart in accordance with embodiments herein.

FIG. 4A illustrates a schematic diagram of a lead being inserted into a septal wall of a heart in accordance with embodiments herein.

FIG. 4B illustrates a schematic diagram of a lead being inserted into a septal wall of a heart in accordance with embodiments herein.

FIG. 4C illustrates a schematic diagram of a lead being inserted into a septal wall of a heart in accordance with embodiments herein.

FIG. 5 illustrates a graph of pacing of a heart over time as a lead is inserted into a septal wall of the heart in accordance with embodiments herein.

FIG. 6 illustrates a flow block diagram of a process for determining when a lead has reached a target depth when penetrating a septal wall during implant in accordance with embodiments herein.

FIG. 7 illustrates a simplified block diagram of an external device in accordance with embodiments herein.

### DETAILED DESCRIPTION

Embodiments set forth herein include methods and systems for continuous monitoring of ECG morphology change during the fixation, or deep septal lead placement, into the septal wall for LBBp. The ECG morphology can provide graphing trends, or set of data values, related to the depth the lead into the septal wall. Taking advantage of this information, a determination is made when a first electrode has reached the mid-septum wall and passed onto the LV septum side wall.

The width of the QRS (WQRSᵢ) in milliseconds (ms) and the peak-to-peak amplitude (Ampkᵢ) in millivolts (mV) can be measured and recorded for the start of lead fixation, on the RV septal side. As the lead is fixated into the septum, new measurements of the QRS width (WQRSₜ) and peak-to-peak amplitude (Ampkₜ) are recorded. In one example, these can be done beat to beat, or alternatively as an average of several beats to get a smoother impedance transition. Both metrics, WQRSₜ & Ampkₜ, are compared to the previous recorded measurement, WQRSₜ₋₁ & Ampkₜ₋₁. In addition, the change in paced morphology can be monitored. In one example, as the lead is screwed into the septum, the notch in lead V1 moves up with a QR pattern. If the previous recording of QRS width is greater than the current and the previous measurement of peak-to-peak amplitude is less than the current, then a determination can be made that the lead is still on the RV side burrowing into the septum, and monitoring continues.

If on the other hand, the current measured peak-to-peak amplitude is greater than the previous recorded measurement and the current measured QRS width is less than the previous measurement, then the lead has passed the mid septal line and is now tunneling into the LV septal side. At this point careful attention in paced morphology of the QRS in lead V1, to the QRS width and peak-to-peak amplitude should be noted, to prevent the implanted lead from perforating into the LV cavity. When the current measured QRS width (WQRSₜ) is within a determined range of the initial recorded impedance (WQRSᵢ), the lead has reached the desired target depth. In one example, a communication such as a message can indicate to stop tunneling further. Alternatively, an algorithm can prompt the user to stop advancing the lead when a significant QRS width increase from the minimum measured QRS width (WQRSₘᵢₙ) has been detected. The QRS width increase can be determined based on absolute QRS width change, percent change, or rate of change. The threshold value for the QRS drop can either be a fixed value or user programmable. The user defined thresholds can be tracked to meet the criteria for deep septal pacing.

Embodiments may be implemented in connection with one or more implantable medical devices (IMDs). Non-limiting examples of IMDs include one or more of implantable leadless monitoring and/or therapy devices, and/or alternative implantable medical devices. For example, the IMD may represent a cardiac monitoring device, pacemaker, cardioverter, cardiac rhythm management device, defibrillator, neurostimulator, leadless monitoring device, leadless pacemaker, and the like.

Additionally or alternatively, the IMD may be a leadless implantable medical device (LIMD) that is directly screwed or otherwise secured to the septal wall with a distal electrode implanted at a target depth within the septal wall. The leadless IMD may include one or more structural and/or functional aspects of the device(s) described in U.S. Pat. No. 9,216,285 “Leadless Implantable Medical Device Having Removable And Fixed Components” and U.S. Pat. No. 8,831,747 “Leadless Neurostimulation Device And Method Including The Same”, which are hereby incorporated by reference. Additionally or alternatively, the IMD may include one or more structural and/or functional aspects of the device(s) described in U.S. Pat. No. 8,391,980 “Method And System For Identifying A Potential Lead Failure In An Implantable Medical Device” and U.S. Pat. No. 9,232,485 “System And Method For Selectively Communicating With An Implantable Medical Device”, which are hereby incorporated by reference.

Additionally or alternatively, the leadless IMD may communicate and coordinate monitoring and/or therapy delivery with a subcutaneous IMD that includes one or more structural and/or functional aspects of the device(s) described in U.S. application Ser. No. 15/973,195, titled “Subcutaneous Implantation Medical Device With Multiple Parasternal-Anterior Electrodes” and filed May 7, 2018; U.S. application Ser. No. 15/973,219, titled “Implantable Medical Systems And Methods Including Pulse Generators And Leads” filed May 7, 2018; U.S. application Ser. No. 15/973,249, titled “Single Site Implantation Methods For Medical Devices Having Multiple Leads”, filed May 7, 2018, which are hereby incorporated by reference in their entireties. Further, one or more combinations of IMDs may be utilized from the above incorporated patents and applications in accordance with embodiments herein.

Additionally or alternatively, the IMD may utilize one or more structural and/or functional aspects of the device(s) described in U.S. Patent Application having Docket No. A15E1059, U.S. patent application Ser. No. 15/084,373, filed Mar. 29, 2016, entitled, “METHOD AND SYSTEM TO DISCRIMINATE RHYTHM PATTERNS IN CARDIAC ACTIVITY,” which is expressly incorporated herein by reference.

Embodiments may be implemented in connection with one or more passive IMDs (PIMDs). Non-limiting examples of PIMDs may include passive wireless sensors used by themselves, or incorporated into or used in conjunction with other implantable medical devices (IMDs) such as cardiac monitoring devices, pacemakers, cardioverters, cardiac rhythm management devices, defibrillators, neurostimulators, leadless monitoring devices, leadless pacemakers, replacement valves, shunts, grafts, drug elution devices, blood glucose monitoring systems, orthopedic implants, and the like. For example, the IMD with an electrode implanted in the septal wall may operate in combination with monitoring additional characteristics such as described in U.S. Pat. No. 9,265,428 entitled “Implantable Wireless Sensor”, U.S. Pat. No. 8,278,941 entitled “Strain Monitoring System and Apparatus”, U.S. Pat. No. 8,026,729 entitled “System and Apparatus for In-Vivo Assessment of Relative Position of an Implant”, U.S. Pat. No. 8,870,787 entitled “Ventricular Shunt System and Method”, and U.S. Pat. No. 9,653,926 entitled “Physical Property Sensor with Active Electronic Circuit and Wireless Power and Data Transmission”, which are all hereby incorporated by reference in their respective entireties.

All references, including publications, patent applications and patents, cited herein are hereby incorporated by reference to the same extent as if each reference were individually and specifically indicated to be incorporated by reference and were set forth in its entirety herein.

### I. Terms and Abbreviations

The terms “cardiac activity signal”, “cardiac activity signals”, “CA signal” and “CA signals” (collectively “CA signals”) are used interchangeably throughout to refer to measured signals indicative of cardiac activity by a region or chamber of interest. The cardiac activity may be normal/healthy or abnormal/arrhythmic. An example of CA signals includes EGM signals. Electrical based CA signals refer to an analog or digital electrical signal recorded by two or more electrodes, where the electrical signals are indicative of cardiac activity.

The terms “beat”, and “cardiac event” are used interchangeably and refer to both normal or abnormal events.

The term “morphology data” as used interchangeably herein refers to any and all characteristics, qualities, features, measurements, etc. of a signal, including a cardiac activity signal. Morphology data can include a set of data values, or sets of data values, related to the cardiac activity signal. Example morphology data can include QRS width, changes in QRS width, average QRS width of a series of QRS widths, a peak-to-peak amplitude, frequency, slope, changes in peak-to-peak amplitude, frequency, or slope, wavelength, or the like. In example embodiments, the morphology data can be utilized with a criteria of interest, including through comparison, to make determinations.

The term “target depth” as used herein may be utilized to describe all depths that place a first electrode proximate a region of interest within the heart's conductive pathways (e.g., the RBB, LBB, Purkinje fibers, etc.) without penetrating the distal end wall of the corresponding chamber. For example, the target depth may include a range of depth through the septal wall, starting with the depth when a first electrode engages the LV septum side of the septal wall to a depth right before the distal end wall proximate the LBB is perforated by the first electrode. The term “depth”, alone, refers to how far into the heart wall (e.g., septal wall) that the first electrode has advanced, whereas the target depth is a desired location of where the first electrode is to be implanted. The target depth in one example may be measured in centimeters (cm), and in one embodiment is in a range between 1 to 1.5 cm into a septal wall.

The term “criteria of interest” when used herein shall mean any and all parameters, characteristics, measurements, settings, rates, percentages, thresholds, etc. that may be utilized to determine the depth of an electrode within a septal wall of the heart. Optionally, criteria of interest can be detected, calculated, obtained, determined, or the like. Example criteria of interest can include, a QRS width, a peak-to-peak amplitude, frequency, slope, mathematical relationship, mathematical model, function, algorithm, or the like related to a parameter, characteristic, measurement, etc.

The terms “processor,” “a processor”, “one or more processors” and “the processor” shall mean one or more processors. The one or more processors may be implemented by one, or by a combination of more than one implantable medical device, a wearable device, a local device, a remote device, a server computing device, a network of server computing devices and the like. The one or more processors may be implemented at a common location or at distributed locations. The one or more processors may implement the various operations described herein in a serial or parallel manner, in a shared-resource configuration and the like.

The term “obtains” and “obtaining”, as used in connection with data, signals, information, and the like, include at least one of i) accessing memory of an external device or remote server where the data, signals, information, etc. are stored, ii) receiving the data, signals, information, etc. over a wireless communications link between the IMD and a local external device, and/or iii) receiving the data, signals, information, etc. at a remote server over a network connection. The obtaining operation, when from the perspective of an IMD, may include sensing new signals in real time, and/or accessing memory to read stored data, signals, information, etc. from memory within the IMD. The obtaining operation, when from the perspective of a local external device, includes receiving the data, signals, information, etc. at a transceiver of the local external device where the data, signals, information, etc. are transmitted from an IMD and/or a remote server. The obtaining operation may be from the perspective of a remote server, such as when receiving the data, signals, information, etc. at a network interface from a local external device and/or directly from an IMD. The remote server may also obtain the data, signals, information, etc. from local memory and/or from other memory, such as within a cloud storage environment and/or from the memory of a workstation or clinician external programmer.

The term “real-time” refers to a time frame contemporaneous with a normal or abnormal episode occurrence. For example, a real-time process or operation would occur during or immediately after (e.g., within minutes or seconds after) a cardiac event, a series of cardiac events, an arrhythmia episode, and the like.

### II. System Overview

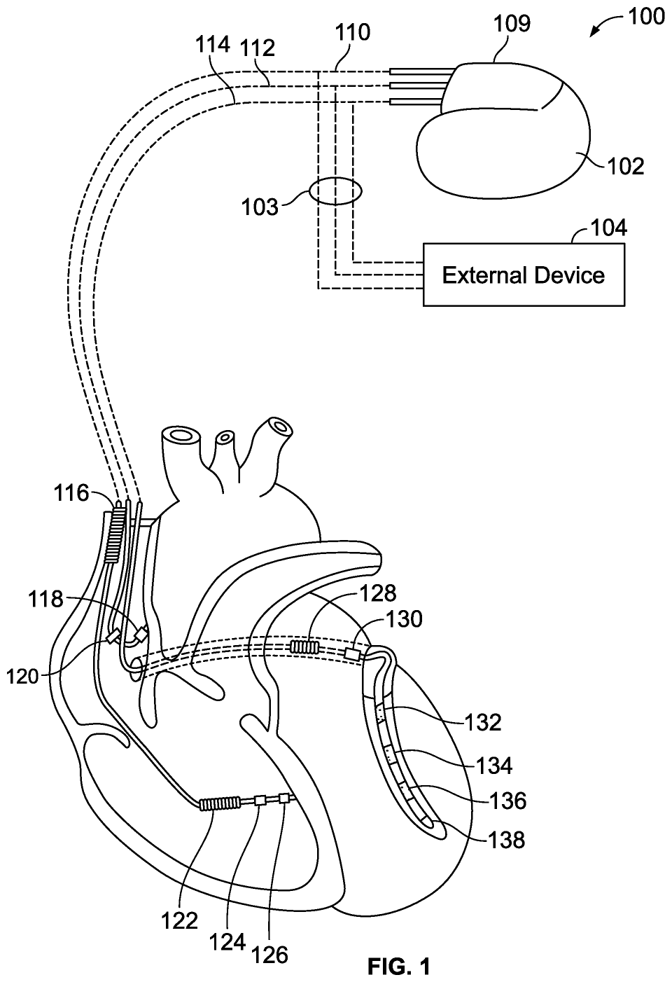

FIG. 1 illustrates an IMD 100 that in one example is a dual-chamber stimulation device capable of treating arrhythmias with stimulation therapy, including cardioversion, defibrillation, anti-tachycardia pacing, HF pacing, and pacing stimulation, as well as capable of detecting heart failure, evaluating its severity, tracking the progression thereof, and controlling the delivery of therapy and warnings in response thereto.

In the example of FIG. 1, the IMD 100 includes a housing 102 that is joined to a header assembly 109 that holds receptacle connectors connected to a right ventricular lead 110, a right atrial lead 112, and a coronary sinus lead 114, respectively. The leads 112, 114 and 110 measure cardiac signals of the heart. While the present embodiment is described in connection with CA signals and heart conditions, it is understood that embodiments may be implemented in connection with other types of biological signals and other types of physiologic and non-physiologic conditions. Additionally, in other example embodiments, a lead may be implanted into a septal wall.

The right atrial lead 112 includes an atrial tip electrode 118 and an atrial ring electrode 120. The coronary sinus lead 114 includes a left atrial ring electrode 128, a left atrial coil electrode 130 and one or more left ventricular electrodes 132-138 (e.g., also referred to as P1, M1, M2 and D1) to form a multi-pole LV electrode combination. The right ventricular lead 110 includes an RV tip electrode 126, an RV ring electrode 124, an RV coil electrode 122, and an SVC coil electrode 116. The leads 112, 114 and 110 detect IEGM signals that are processed and analyzed as described herein. The leads 112, 114 and 110 also delivery therapies as described herein.

During implantation, an external device 104 is connected to one or more of the leads 112, 114 and 110 through temporary inputs 103. The inputs 103 of the external device 104 receive IEGM signals from the leads 112, 114 and 110 during implantation and display the IEGM signals to the physician on a display. Hence, the external device 104 receives the IEGM cardiac signals through telemetry circuit inputs. The physician or another user controls operation of the external device 104 through a user interface. While the example embodiment of FIG. 1 illustrates an IMD 100 that includes leads, such embodiment is for exemplary purposes only, in other example embodiments, the IMD may be a leadless IMD.

Embodiments may utilize one or more leadless IMD that are implanted at various locations within the heart, such as in the RA, RV, LA, LV. A leadless IMD may be implanted in at an RV apical septum where the target depth represents a depth within the apical septum at which the conductive pathway is located (e.g., the RBB, Purkinje fibers). Additionally or alternatively, a leadless IMD may be implanted in at an LV apical septum where the target depth represents a depth within the apical septum at which the conductive pathway is located (e.g., the LBB, Purkinje fibers).

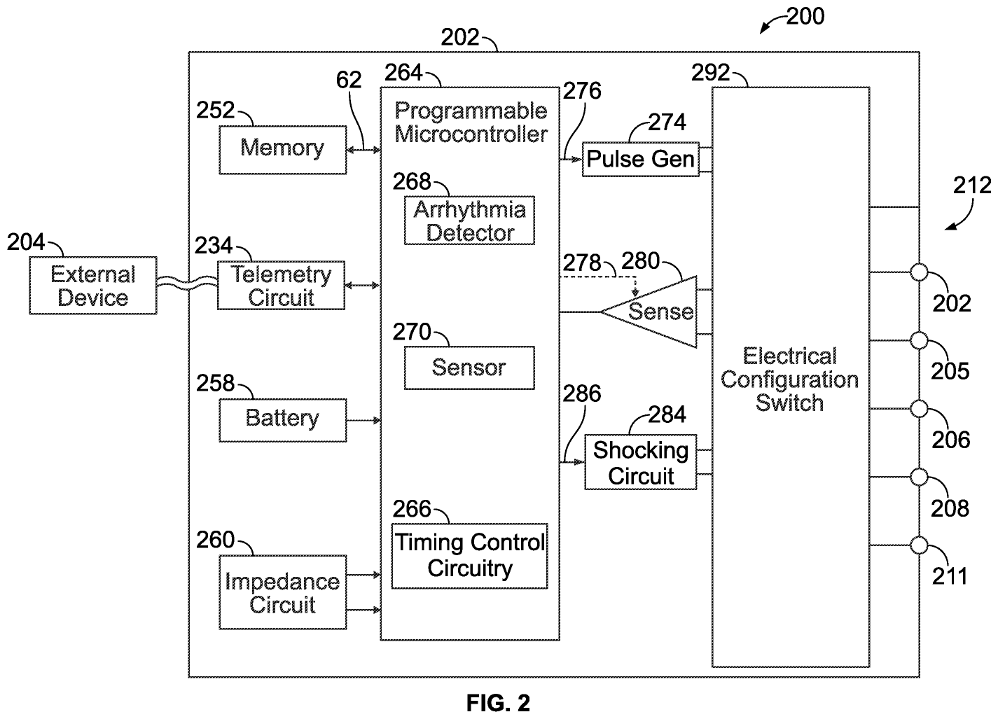

FIG. 2 illustrates an example block diagram of a IMD 200 that is implanted into the patient as part of the implantable cardiac system. While described as an implantable cardiac system, this is for exemplary purposes only, and other IMDs are contemplated. In this embodiment, the IMD 200 may be implemented as a full-function biventricular pacemaker, equipped with both atrial and ventricular sensing, and pacing circuitry for four chamber sensing and stimulation therapy (including both pacing and shock treatment). The pacing circuitry in one example includes HF pacing. Optionally, the IMD 200 may provide full-function cardiac resynchronization therapy. Alternatively, the IMD 200 may be implemented with a reduced set of functions and components. For instance, the monitoring device may be implemented without ventricular sensing and pacing.

The IMD 200 has a housing 201 to hold the electronic/computing components. The housing 201 (which is often referred to as the “can”, “case”, “encasing”, or “case electrode”) may be programmably selected to act as the return electrode for certain stimulus modes. Housing 201 further includes a connector (not shown) with a plurality of terminals 202, 205, 206, 208, and 211 for electrodes 212. The type and location of each electrode may vary. For example, the electrodes may include various combinations of ring, tip, coil and shocking electrodes and the like.

The IMD 200 also includes a telemetry circuit 234 that as a primary function allows intracardiac electrograms and status information relating to the operation of the IMD 200 (as contained in the microcontroller 264 or memory 252) to be sent to the external device 204 through the established communication link 250. Specifically, the telemetry circuit wirelessly communicates with at least one of the external device 204 or second implanted device.

The IMD 200 also includes a programmable microcontroller 264 that controls various operations of the IMD 200. Microcontroller 264 includes a microprocessor (or equivalent control circuitry), RAM and/or ROM memory, logic and timing circuitry, state machine circuitry, and I/O circuitry. The IMD 200 further includes a first chamber pulse generator 274 that generates stimulation pulses for delivery by one or more electrodes coupled thereto. The pulse generator 274 is controlled by the microcontroller 264 via control signal 276. The pulse generator 274 is coupled to the select electrode(s) via an electrode configuration switch 292, which includes multiple switches for connecting the desired electrodes to the appropriate I/O circuits, thereby facilitating electrode programmability. The switch 292 is controlled by a control signal 286 from the microcontroller 264.

Microcontroller 264 is illustrated to include timing control circuitry 266 to control the timing of the stimulation pulses (e.g., pacing rate, atrio-ventricular (AV) delay, atrial interconduction (A-A) delay, or ventricular interconduction (V-V) delay, etc.). Microcontroller 264 also has an arrhythmia detector 268 for detecting arrhythmia conditions. Although not shown, the microcontroller 264 may further include other dedicated circuitry and/or firmware/software components that assist in monitoring various medical conditions, including of the patient's heart and managing pacing therapies, including HF pacing.

The IMD 200 also includes one or more sensors 270. The one or more sensors 270 can include physiological sensors that detect characteristics associated with the heart of the patient. Alternatively, the one or more sensors 270 can be environmental sensors that detect characteristics associated with the environment of the patient. In one example, the sensor 270 is an accelerometer that obtains location information with respect to gravitational force while the IMD 200 collects cardiac activity signals in connection with multiple cardiac beats. The microcontroller 264 may utilize the signals from the accelerometer to determine a change in posture, change in activity, or the like. While the sensor 270 shown as being included within the housing 202, the sensor 270 may be external to the housing 202, yet still, be implanted within or carried by the patient.

The IMD 200 is further equipped with a communication modem (modulator/demodulator) to enable wireless communication with other devices, implanted devices, and/or external devices. The IMD 200 also includes sensing circuitry 280 selectively coupled to one or more electrodes that perform sensing operations, through the switch 292, to detect the presence of cardiac activity.

The output of the sensing circuitry 280 is connected to the microcontroller 264 which, in turn, triggers or inhibits the pulse generator 274 in response to the absence or presence of cardiac activity. The sensing circuitry 280 receives a control signal 278 from the microcontroller 264 for purposes of controlling the gain, threshold, polarization charge removal circuitry (not shown), and the timing of any blocking circuitry (not shown) coupled to the inputs of the sensing circuitry 280. The sensing circuitry 280 can also be utilized to provide cardiac activity signals during the implanting of a lead through a septal wall. By using changes in morphology of the cardiac activity signals, determinations can be made related to the location of the lead within the septal wall.

The microcontroller 264 is also coupled to a memory 252 by a suitable data/address bus 262. The programmable operating parameters used by the microcontroller 264 are stored in memory 252 and used to customize the operation of the IMD 200 to suit the needs of a particular patient. Such operating parameters define, for example, pacing pulse amplitude, pulse duration, electrode polarity, rate, sensitivity, automatic features, HF pacing features, arrhythmia detection criteria, and the amplitude, waveshape and vector of each shocking pulse to be delivered to the patient's heart within each respective tier of therapy. Such shocking pulse can be an LV shock, MV shock, HV shock, etc.

A battery 258 provides operating power to all of the components in the IMD 200. The IMD 200 further includes an impedance circuit 260, which can be used for many things, including: lead impedance surveillance during the acute and chronic phases for proper lead positioning or dislodgement; detecting operable electrodes and automatically switching to an operable pair if dislodgement occurs; measuring thoracic impedance for determining shock thresholds; detecting when the device has been implanted; measuring stroke volume; and detecting the opening of heart valves; and so forth. The impedance circuit 260 is coupled to the switch 292 so that any desired electrode may be used. The IMD 200 can be operated as an implantable cardioverter/defibrillator (ICD) device, which detects the occurrence of an arrhythmia and automatically applies an appropriate electrical shock therapy to the heart aimed at terminating the detected arrhythmia. To this end, the microcontroller 264 further controls a shocking circuit 284 by way of a control signal 286, including for LV shocks, MV shocks, HV shocks, etc.

The physiologic sensor may be implemented as an accelerometer and may be implemented utilizing all or portions of the structural and/or functional aspects of the methods and systems described in U.S. Pat. No. 6,937,900, titled “AC/DC Multi-Axis Accelerometer for Determining A Patient Activity and Body Position;” U.S. application Ser. No. 17/192961, filed 3/5/2021, (Attorney docket 13-0397US1) (client docket 13967USO1), titled “SYSTEM FOR VERIFYING A PATHOLOGIC EPISODE USING AN ACCELEROMETER”; U.S. application Ser. No. 16/869733, filed 5/8/2020, (Attorney docket 13-0396US1) (client docket 13964USO1), titled “METHOD AND DEVICE FOR DETECTING RESPIRATION ANOMALY FROM LOW FREQUENCY COMPONENT OF ELECTRICAL CARDIAC ACTIVITY SIGNALS;” U.S. application Ser. No. 17/194354, filed 3/8/2021, (Attorney docket 13-0395US1) (client docket 13949USO1), titled “METHOD AND SYSTEMS FOR HEART CONDITION DETECTION USING AN ACCELEROMETER,” the complete subject matter which is expressly incorporated herein by reference.

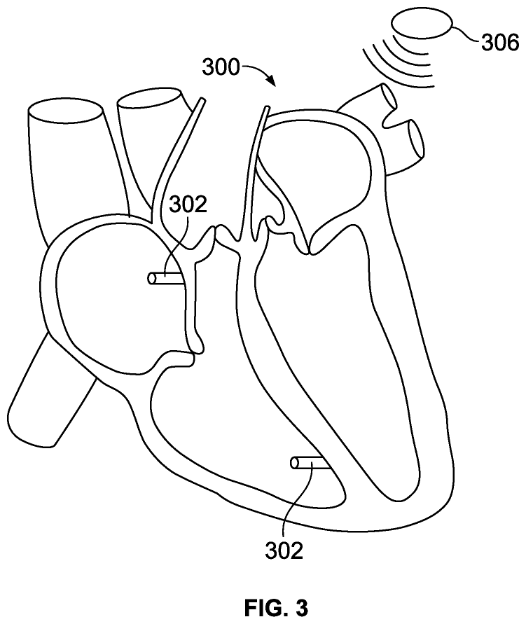

FIG. 3 illustrates a pictorial view depicting an embodiment of an implantable system 300 that may be utilized to deliver various types of therapy and/or monitor physiologic conditions in accordance with embodiments herein. The system 300 comprises one or more implantable medical devices (IMD) 302. As described further below, the IMD 302 comprises a housing, multiple electrodes coupled to the housing, and a pulse generator hermetically contained within the housing and electrically coupled to the electrodes. The pulse generator may be configured for sourcing energy internal to the housing, generating, and delivering electrical pulses to the electrodes. A controller can also be hermetically contained within the housing as part of the pulse generator and communicatively coupled to the electrodes. The controller can control, among other things, recording of physiologic characteristics of interest and/or electrical pulse delivery based on the sensed activity.

In the example of FIG. 3, a pair of IMDs 302 are illustrated to be located in different first and second chambers of the heart. For example, one IMD 302 is located in the right atrium (RA), while a second IMD 302 is located in the right ventricle (RV). Optionally, the IMD 302 located in the RA is advanced to a target depth to locate a distal, or first electrode, at the HIS or AV node. In one example, the IMD 302 is advanced by screwing the IMD 302 to the target depth. Alternatively, in another example, the IMD 302 located in the RV can be advanced, screwed, etc. to a target depth with the distal, or first electrode at one of the RBB, or LBB.

The IMDs 302 coordinate the operation therebetween based in part on information conveyed between the IMDs 302 during operation. The information conveyed between the IMDs 302 may include, among other things, physiologic data regarding activity occurring in the corresponding local chamber. For example, the atrial IMD 302 may perform sensing and pacing operations in the right atrium, while the ventricular IMD 302 may perform sensing and pacing operations in the right ventricle. The physiologic data conveyed between the atrial and ventricular IMDs 302 includes, among other things, the detection of sensed intrinsic local events (e.g. sensed atrial events or sensed ventricular events). The physiologic data also includes paced local events (e.g. paced atrial events or paced ventricular events). Additionally or alternatively, the information conveyed between the atrial and ventricular IMDs 302 may include device related information, such as synchronization information, oscillator clock timing information, battery status, quality information regarding received signals and the like. Additionally or alternatively, the physiologic data may include other information.

While the IMDs 302 are located in the right atrium and ventricle, optionally, the IMDs 302 may be located in other chamber combinations of the heart, as well as outside of the heart. Optionally, the IMDs 302 may be located in a blood pool without directly engaging local tissue. Optionally, the IMDs 302 may be implemented solely to perform monitoring operations, without delivery of therapy. For example, an IMD 302 may be a cardiac monitoring device that is located outside of, but in relatively close proximity to, the heart. As another example, one or more IMDs 302 may represent a subcutaneous implantable device located in a subcutaneous pocket and configured to perform monitoring and/or deliver therapy. As another example, one or more IMDs 302 may be configured to perform neural stimulation. The IMD 302 is located proximate to nerve tissue of interest (e.g., along the spinal column, dorsal root, brainstem, within the brain, etc.). The IMD 302 may be configured to perform monitoring of neural activity, without delivering neural stimulation. Optionally, the IMD 302 may not require tissue contact to monitor and/or deliver therapy. For example, blood pressure may be measured with or without direct tissue contact. The IMDs 302 may also receive communication, data, information, signals, etc. from an external device 306.

In the example of FIG. 3, the IMDs 302 represent leadless devices in which the electrodes are located directly on the housing of the device, without a lead extending from the device housing. Optionally, the IMDs 302 may be implemented with leads, where the conducted communication occurs between one or more electrodes on the lead and/or on the housing. Examples of other IMDs that may be configured to implement the conducted communication embodiments described herein are described in U.S. Pat. No. 9,168,383, issued Oct. 27, 2015, and titled “LEADLESS CARDIAC PACEMAKER WITH CONDUCTED COMMUNICATION,” the complete subject matter of which is incorporated by reference in its entirety.

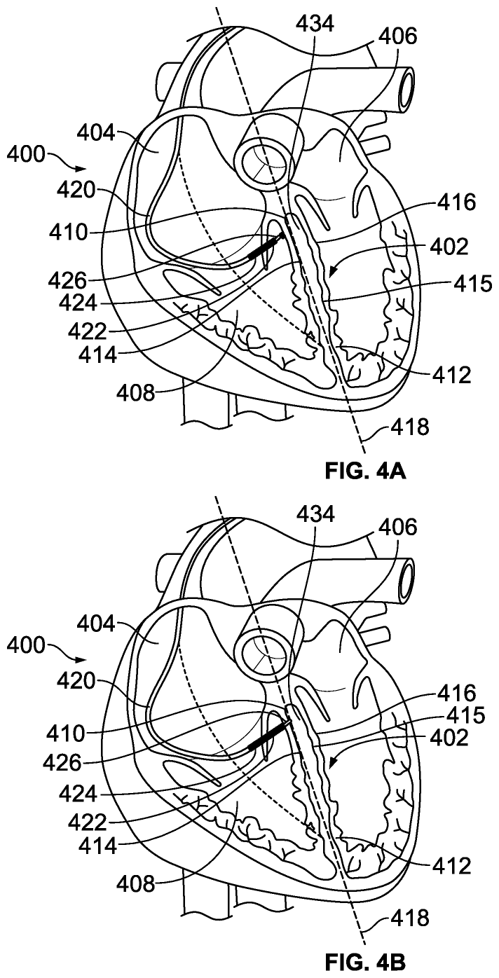

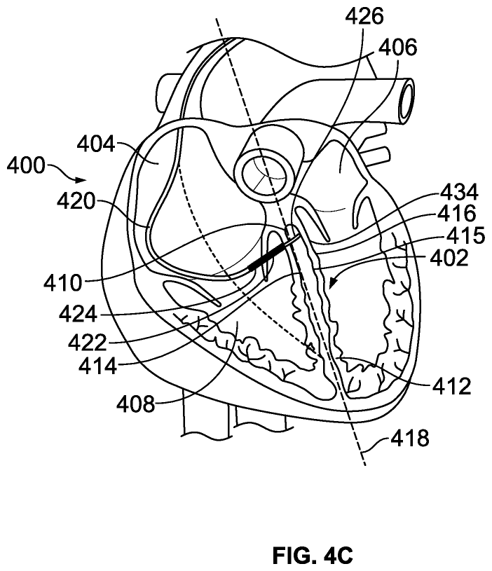

FIGS. 4A-4C illustrate the advancement of a lead into a heart 400 for placement of the lead 403 in and through a septal wall 402 in real-time. The heart 400 includes a right atrium 404, left atrium 406, left ventricle 408, and right ventricle with the septal wall 402 centrally located extending from a HIS 410 to an apex 412 and separating the right chambers of the heart 400 from the left chambers of the heart 400. The septal wall 402 includes a RV septum side wall 414 on the right side of a mid-septum wall 415, and a LV septum side wall 416 on the left side of the mid-septum wall 415. A mid septal line 418 extends through the mid-septum wall 415 and represents the midway line through the septal wall 402.

In the example embodiment of FIGS. 4A-4C, a catheter 420 is illustrated that includes a lead 403 of an IMD that extends from a proximal end 424 to a distal end 426. While a lead 403 is illustrated, in other example embodiments, a leadless IMD can similarly be inserted and advanced through the septal wall 402. Additionally, while in the example embodiment, the process and methodology are utilized to determine a depth of the lead 403 during advancement of the lead 403 into a septal wall 402, in other example embodiments, the process and methodology is utilized to determine the depth of a lead 403 through a different portion of the heart. In one example, the process and method are utilized to determine the depth of a lead 403 proximate an outer chamber wall, including implanting of a leadless device in an outer chamber wall, apical septum, and the like.

The septal wall component 434 includes a proximal portion of the RV septum side wall 414 and extends to a distal portion of the LV septum side wall 416. The LV/LA chamber blood component 442 is representative of the blood flowing through either one of or both the left ventricle and left atrial. As an example, depending on the location of the lead, the morphology of a QRS wave of the heart varies.

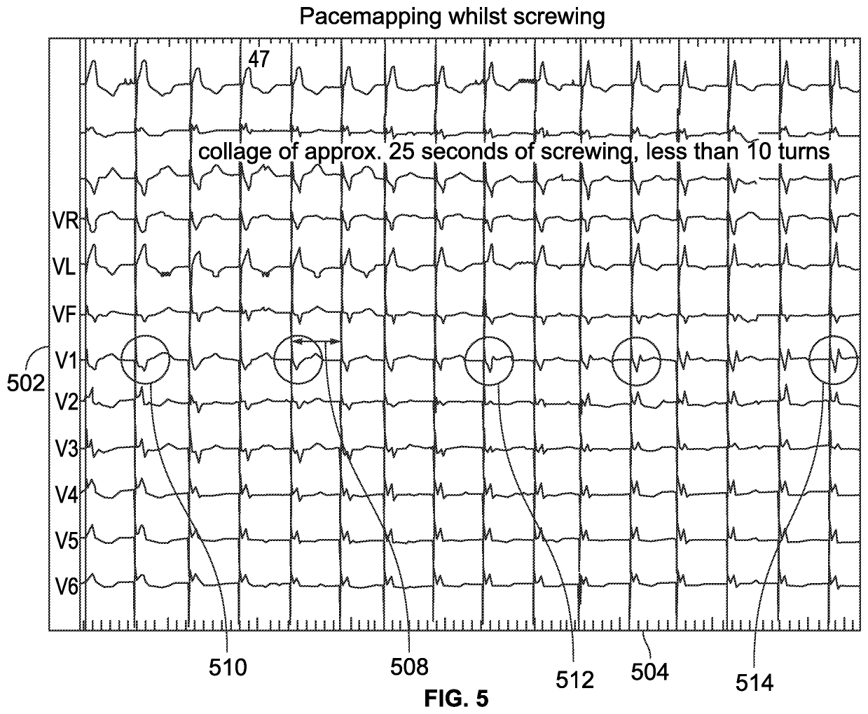

FIG. 5 illustrates a graph of heart pacing 502 over time 504 as the lead 403 from FIGS. 4A-4C is screwed into the septal wall 402. In a first position (FIG. 4A) when the lead 403 is first engaging the RV septum side wall 414 and is not proximate the LV septum side wall 416 the QRS width 506 and peak-to-peak amplitudes 508 show a first morphology 510. As the lead continues to be screwed into the septal wall 402 a second position (FIG. 4B) can be reached after the lead has been advanced through the RV septum side wall 414 into the mid-septum wall 415 but is still not proximate the LV septum side wall 416. In one example, the lead is pushed through the RV septum side wall 414. Alternatively, the lead is rotated, or screwed through the RV septum side wall.

At the second position (FIG. 4B), the lead 403 engages the mid-septum wall 415, and a second morphology 512 is provided. As illustrated, the morphology, including the QRS width 506 and peak-to-peak amplitudes have significantly changed based on the location of the lead 403. Because these changes are noticeable to one inserting the lead 403 a determination can be made of the location of the lead.

Next, the lead 403 advances to a third position (FIG. 4C), with the lead advancing through a distal portion of the mid-septum wall 415 and the lead 403 is proximate the LBB. The third position represents the desired location of the lead 403 providing therapy. Consequently, during the implant, the clinician desires to stop advancing the lead 403 at this location. In the third position, a third morphology 514 is provided, again with an altered morphology, including a different QRS width 506 and peak-to-peak amplitude 508 compared to the first position and second position. Because of the distinct morphology changes, a clinician can be utilized the change in the morphology to determine the location of the lead 403 in the septal wall 402. To this end, the clinician can be assured that the lead 403 is proximate the LBB without perforating through the LV septum side wall 416 and into a chamber of the heart 400.

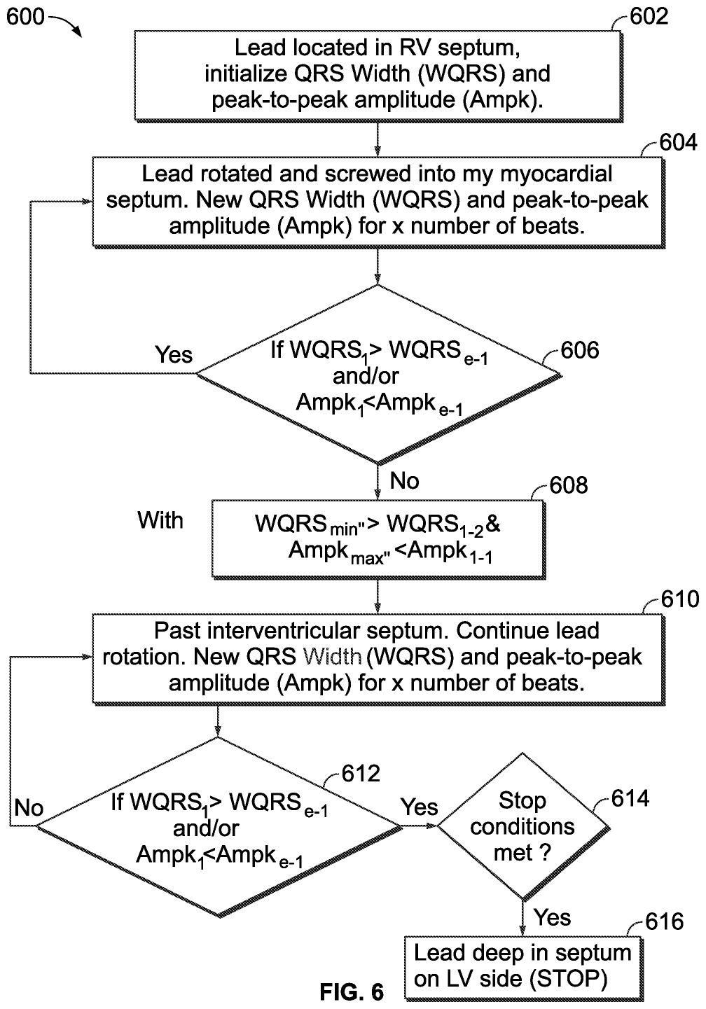

FIG. 6 illustrates a flow block diagram of a process 600 for determining when a lead has reached a target depth when penetrating a septal wall during implant. In one example, the lead of FIGS. 4A-4C are utilized for implementing the process. The process can be implemented to implant a lead based device, a leadless device, an IMD, an ICD, an ICM, or the like. Additionally or alternatively, the process of FIG. 6 may be implemented utilizing all or portions of the structural and/or functional aspects of the methods and systems described in co-pending application No. 17/007,696, filed Aug. 31, 2020, and titled “SYSTEMS AND METHODS FOR IMPLANTING A MEDICAL DEVICE USING AN ACTIVE GUIDEWIRE”, the complete subject matter of which is expressly incorporated herein by reference in its entirety.

At 602, a lead is inserted into the right ventricle (RV) septum and cardiac activity signals are continuously obtained by a heart monitoring device. From the cardiac activity signals, morphology data, including sets of data values begin to be obtained. In one example, the sets of data values include a graph of consecutive heartbeats obtained from a heart monitoring device. The sets of data values can also include QRS width(s) (WQRS) and peak-to-peak amplitude(s). These can include a first, or initial, QRS width, and a first, or initial peak-to-peak amplitude. As used herein, the first QRS width, first peak-to-peak amplitude, etc. do not have to be related to the first heartbeat obtained once the lead is inserted into the RV septum. Instead, the term “first” only relates to an initial reading that is utilized to make a calculation or determination to a change compared to a later (second) value. In addition, the later, or second value, does not have to necessarily be a consecutive beat to the first heartbeat utilized. In opposite, most likely, the “second” heartbeat utilized for determining a second QRS width, second peak-to-peak amplitude, etc. will not be a from a consecutive heartbeat with the first heartbeat. Instead, numerous heartbeats will exist between the initial, or first heartbeat and the second heartbeat. Thus, first and second only reference a comparison between two heartbeats where the second (and accompanying morphology data) is obtained at a later time than the first. Still, the first morphology data is obtained in close proximity to the time the lead first enters the RV septum.

At 604, the lead moves through the septum. In one example, the lead is twisted, screwed, turned, etc. to facilitate insertion into the septum. As the lead moves through the septum, cardiac activity signals, along with their accompanying morphology data for each and every consecutive heartbeat is obtained and continuously compared to the initial, or first morphology data. In particular, at 606, the comparison between the most recent morphology data and the initial morphology data is utilized to determine if a change in the data values satisfy a criteria of interest. By comparing the most recent morphology data, a determination can be made regarding whether a relation exists that satisfies the criteria of interest. For example, a criteria of interest may be a change in QRS width reaching a change in QRS width value that indicates the lead has moved through the RV septum and into the mid-septum wall. In one example, numerous sets of data values are obtained from numerous patients having several personal characteristics that are like the patient. These personal characteristics can include gender, age, height, weight, blood pressure, medical conditions, previous medical procedures, or the like. The previous data sets can be averaged, compared, utilized with a model, used in association with artificial intelligence, etc. to determine an expected change in criteria of interest that indicates the location of the lead. In one example, the criteria of interest is the QRS width of a cardiac activity signal, and when the change in the QRS width reaches a QRS width threshold, an indication is provided that the lead has reached the mid-septum. Alternatively, the criteria of interest can be a peak-to-peak amplitude of the cardiac activity signal, and when the change in the peak-to-peak amplitude reaches a peak-to-peak amplitude threshold, an indication is provided that the lead has reached the mid-septum.

In one example, the criteria of interest is determined by analyzing trends in morphology data and information of numerous patients. The patient characteristics, cardiac characteristics, etc. of numerous other patients are analyzed to determine trends in changes of morphology that occurs as a lead is inserted through a septal wall. In one example, an algorithm, and in one example, an artificial intelligence algorithm can be utilized to make comparisons between patients to provide weights to different patient characteristics, and morphologies as they correspond to the insertion of the lead. In this manner, a patient's age, weight, heart health, preexisting conditions, etc. along with peak-to-peak amplitude, frequency, QRS width, or the like can all be input into an algorithm and provided with differing weights to predict where the location of a lead is within the septal wall based on the current morphology of a patient. As more information and implants are performed, artificial intelligence algorithm can adjust the weights more accurately make determinations regarding the location of the lead during a procedure. For example, if in 95% in all patients, regardless of age or health conditions, when the QRS width decreases in two consecutive beat the mid-septum is reached, then a trend is presented that two straight beat with QRS width decreases indicates the mid-septum has been reached. In one example, because of the trend, when two consecutive beats with QRS widths are provided, a notification that the mid-septum is reached can be automatically provided. Alternatively, a very strong weight can be provided to this characteristics; however, other characteristics can still be provided weights to ensure the instance is not one of the 5% of the times the mid-septum is not reached. In this manner, trends in the morphology information can be utilized to make determination related to the location of the lead during a procedure.

If at 606 a determination Yes is made that the lead has not reached the mid-septum, then the clinician continues inserting the lead through the septum, and morphology data continues to be obtained and compared to the initial morphology data. However, if at 606, a determination No is made that the lead has reached the mid-septum, it means that at 608 the threshold values of the criteria of interest has been reached. At this time, the threshold criteria of interest becomes the new initial, or first criteria of interest. For example, if the criteria of interest is the QRS width, the QRS width that results in the change in QRS width to reach the QRS width threshold when compared to the initial QRS width, now becomes the new initial QRS width. Similarly, when the criteria of interest is the peak-to-peak amplitude, then the peak-to-peak amplitude that results in the change in peak-to-peak amplitude to reach the peak-to-peak amplitude threshold when compared to the initial peak-to-peak amplitude becomes the new initial peak-to-peak amplitude.

At 610, utilizing the new initial criteria of interest from 608, the cardiac activity signals continue to by monitored and morphology data obtained as the clinician continues to insert the lead through the mid-septum. At 612, a determination is continuously made regarding whether a change in the criteria of interest reaches a threshold change. Similar to 606, the determination regarding the change in the criteria (e.g., QRS width, peak-to-peak amplitude, etc.) is made as a result of morphology data obtained from other patients, computer-based models, or the like. Then, if the threshold value is reached, at 614 a determination is made whether stop conditions are met. In particular, once the threshold is reached, an indication, prompt, sound, alert, or the like is provided to the clinician to stop moving the lead because the lead is in the LBB. However, an additional check is provided to verify that the lead is within the LBB and not still in the mid-septum. In particular, the method provides additional safety, to prevent perforation of the LV septum wall during implant. If the LBB has not been reached, then the procedure continues with clinician continuing insertion and monitoring. If the LBB has been reached, at 616 the lead is determined to be at the correct location, and insertion complete.

In Example embodiments the stop conditions can include when the change in QRS width is greater than 10% (e.g., (WQRSₜ₁-WQRSₜ₂)/WQRSₜ₁ X 100=+/−10%; where WQRSₜ₁ is a QRS width at a first time and WQRSₜ₂ is a QRS width at a second time). Alternatively, the stop condition can be based on a peak amplitude threshold (e.g., Ampkₘₐₓ−Ampkₜ=threshold 1; where Ampkₘₐₓ is a maximum peak amplitude and Ampkₜ is a peak amplitude at a given time). In one example the peak amplitude is measured in mV (milli Volts). In another example, the stop condition can be when the percent change in QRS width meets a threshold (e.g., (WQRSₘₐₓ−WQRSₜ₋₁)/WQRSₜ₋₁=threshold2; wherein WQRSₘₐₓ is the maximum QRS width, WQRSₜ₋₁ is a QRS width at a given time; and the threshold2 is a percentage such as 10%). In yet another example, the stop condition can be based on the percent change in amplitude at a first time compared to the peak amplitude at a second time being less than a threshold change (e.g., (Ampkₜ−Ampkₜ₋₁)/Ampkₜ₋₁<threshold 3; wherein Ampkₜ is the peak amplitude at a first time, Ampkₜ₋₁ is the peak amplitude at a second time, and the threshold is a percentage such as 2%). In addition, the stop condition can be any combination of these conditions, including all of them being met.

By utilizing this process, an additional check is provided for the clinician to prevent perforation of the LV septum. Because the heart and cardiac activity signals are already being monitored during such an operation, very few modifications are required to provide the additional monitoring and safety. As such, the method and system are inexpensive and easy to implement.

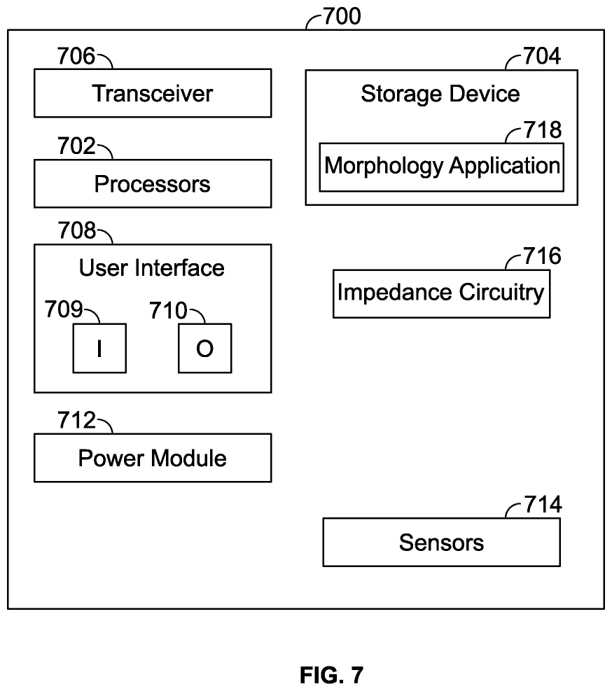

FIG. 7 illustrates a simplified block diagram of an external device 600. In one example, the external device 700 can be the external device 104 of FIG. 1, external device 204 of FIG. 2, an external device utilized in association with another IMD, including the leadless IMD of FIG. 3, or the like.

The external device 700 includes components such as one or more processors 702 (e.g., a microprocessor, microcomputer, application-specific integrated circuit, etc.), one or more local storage medium (also referred to as a memory portion) 704, one or more transceivers 706, a user interface 708 which includes one or more input devices 709 and one or more output devices 710, a power module 712, one or more sensors 714, and impedance circuit 716. All of these components can be operatively coupled to one another, and can be in communication with one another, by way of one or more internal communication links, such as an internal bus.

The local storage medium 704 can encompass one or more memory devices of any of a variety of forms (e.g., read only memory, random access memory, static random-access memory, dynamic random-access memory, etc.) and can be used by the processor 702 to store and retrieve data. The data that is stored by the local storage medium 704 can include, but not be limited to, CA signals, acceleration signals, impedance data, sets of data values, algorithms, applications, or the like. The local storage medium 704 can also include executable code that controls basic functions of the external device 700, such as interaction among the various components, communication with IMDs via the transceiver 706 and storage and retrieval of applications and context data to and from the local storage medium 704. The transceiver 706 may provide wire communication, wireless communication, cellular communication, over the air communication, network communication, electronic communication, a combination thereof, or the like.

In one example embodiment, the local storage medium includes a morphology application 718. The morphology application 718 obtains morphology data related to cardiac activity signals. This morphology data can include criteria of interest such as QRS width, peak-to-peak amplitude, or the like. The morphology application 718 can be utilized in association with the insertion of a lead within the septum wall to locate the lead in the LV septum wall without perforating the LV septum wall. The morphology application can compare criteria of interest (QRS width, peak-to-peak amplitude, etc.) to initial criteria of interest to provide indications to a clinician when the lead has reached the mid-septum and LV septum. By comparing the criteria of interest to determine when threshold changes occur in the criteria of interest, an additional indication is provided of the location of the lead within the septum wall. In example embodiments, the indication that the lead has reached the LV septum wall and is appropriated located can include a light indicator such as a light emitting diode indicator, sound indication, haptic indicator, graphical representation on an output such as a screen, or the like. In each instance, the clinician is alerted that the lead has reached the desired location. In addition, different indicators may be provided to alert the clinician that the mid-septum wall has been reach compared to the LV septum wall.

### Closing

The various methods as illustrated in the Figures and described herein represent exemplary embodiments of methods. The methods may be implemented in software, hardware, or a combination thereof. In various of the methods, the order of the steps may be changed, and various elements may be added, reordered, combined, omitted, modified, etc. Various of the steps may be performed automatically (e.g., without being directly prompted by user input) and/or programmatically (e.g., according to program instructions).

Various modifications and changes may be made as would be obvious to a person skilled in the art having the benefit of this disclosure. It is intended to embrace all such modifications and changes and, accordingly, the above description is to be regarded in an illustrative rather than a restrictive sense.

Various embodiments of the present disclosure utilize at least one network that would be familiar to those skilled in the art for supporting communications using any of a variety of commercially-available protocols, such as Transmission Control Protocol/Internet Protocol (“TCP/IP”), User Datagram Protocol (“UDP”), protocols operating in various layers of the Open System Interconnection (“OSI”) model, File Transfer Protocol (“FTP”), Universal Plug and Play (“UpnP”), Network File System (“NFS”), Common Internet File System (“CIFS”) and AppleTalk. The network can be, for example, a local area network, a wide-area network, a virtual private network, the Internet, an intranet, an extranet, a public switched telephone network, an infrared network, a wireless network, a satellite network, and any combination thereof.

In embodiments utilizing a web server, the web server can run any of a variety of server or mid-tier applications, including Hypertext Transfer Protocol (“HTTP”) servers, FTP servers, Common Gateway Interface (“CGI”) servers, data servers, Java servers, Apache servers and business application servers. The server(s) also may be capable of executing programs or scripts in response to requests from user devices, such as by executing one or more web applications that may be implemented as one or more scripts or programs written in any programming language, such as Java®, C, C# or C++, or any scripting language, such as Ruby, PHP, Perl, Python or TCL, as well as combinations thereof. The server(s) may also include database servers, including without limitation those commercially available from Oracle®, Microsoft®, Sybase® and IBM® as well as open-source servers such as MySQL, Postgres, SQLite, MongoDB, and any other server capable of storing, retrieving, and accessing structured or unstructured data. Database servers may include table-based servers, document-based servers, unstructured servers, relational servers, non-relational servers, or combinations of these and/or other database servers.

The environment can include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (“SAN”) familiar to those skilled in the art. Similarly, any necessary files for performing the functions attributed to the computers, servers or other network devices may be stored locally and/or remotely, as appropriate. Where a system includes computerized devices, each such device can include hardware elements that may be electrically coupled via a bus, the elements including, for example, at least one central processing unit (“CPU” or “processor”), at least one input device (e.g., a mouse, keyboard, controller, touch screen or keypad) and at least one output device (e.g., a display device, printer, or speaker). Such a system may also include one or more storage devices, such as disk drives, optical storage devices and solid-state storage devices such as random access memory (“RAM”) or read-only memory (“ROM”), as well as removable media devices, memory cards, flash cards, etc.

Such devices also can include a computer-readable storage media reader, a communications device (e.g., a modem, a network card (wireless or wired), an infrared communication device, etc.) and working memory as described above. The computer-readable storage media reader can be connected with, or configured to receive, a computer-readable storage medium, representing remote, local, fixed and/or removable storage devices as well as storage media for temporarily and/or more permanently containing, storing, transmitting, and retrieving computer-readable information. The system and various devices also typically will include a number of software applications, modules, services, or other elements located within at least one working memory device, including an operating system and application programs, such as a client application or web browser. It should be appreciated that alternate embodiments may have numerous variations from that described above. For example, customized hardware might also be used and/or particular elements might be implemented in hardware, software (including portable software, such as applets) or both. Further, connection to other computing devices such as network input/output devices may be employed.

Various embodiments may further include receiving, sending, or storing instructions and/or data implemented in accordance with the foregoing description upon a computer-readable medium. Storage media and computer readable media for containing code, or portions of code, can include any appropriate media known or used in the art, including storage media and communication media, such as, but not limited to, volatile and non-volatile, removable and non-removable media implemented in any method or technology for storage and/or transmission of information such as computer readable instructions, data structures, program modules or other data, including RAM, ROM, Electrically Erasable Programmable Read-Only Memory (“EEPROM”), flash memory or other memory technology, Compact Disc Read-Only Memory (“CD-ROM”), digital versatile disk (DVD) or other optical storage, magnetic cassettes, magnetic tape, magnetic disk storage or other magnetic storage devices or any other medium which can be used to store the desired information and which can be accessed by the system device. Based on the disclosure and teachings provided herein, a person of ordinary skill in the art will appreciate other ways and/or methods to implement the various embodiments.

The specification and drawings are, accordingly, to be regarded in an illustrative rather than a restrictive sense. It will, however, be evident that various modifications and changes may be made thereunto without departing from the broader spirit and scope of the invention as set forth in the claims.

Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the invention to the specific form or forms disclosed, but on the contrary, the intention is to cover all modifications, alternative constructions and equivalents falling within the spirit and scope of the invention, as defined in the appended claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including” and “containing” are to be construed as open-ended terms (i.e., meaning “including, but not limited to,”) unless otherwise noted. The term “connected,” when unmodified and referring to physical connections, is to be construed as partly or wholly contained within, attached to, or joined together, even if there is something intervening. Recitation of ranges of values herein are merely intended to serve as a shorthand method of referring individually to each separate value falling within the range, unless otherwise indicated herein and each separate value is incorporated into the specification as if it were individually recited herein. The use of the term “set” (e.g., “a set of items”) or “subset” unless otherwise noted or contradicted by context, is to be construed as a nonempty collection comprising one or more members. Further, unless otherwise noted or contradicted by context, the term “subset” of a corresponding set does not necessarily denote a proper subset of the corresponding set, but the subset and the corresponding set may be equal.

Operations of processes described herein can be performed in any suitable order unless otherwise indicated herein or otherwise clearly contradicted by context. Processes described herein (or variations and/or combinations thereof) may be performed under the control of one or more computer systems configured with executable instructions and may be implemented as code (e.g., executable instructions, one or more computer programs or one or more applications) executing collectively on one or more processors, by hardware or combinations thereof. The code may be stored on a computer-readable storage medium, for example, in the form of a computer program comprising a plurality of instructions executable by one or more processors. The computer-readable storage medium may be non-transitory.

All references, including publications, patent applications and patents, cited herein are hereby incorporated by reference to the same extent as if each reference were individually and specifically indicated to be incorporated by reference and were set forth in its entirety herein.

It is to be understood that the subject matter described herein is not limited in its application to the details of construction and the arrangement of components set forth in the description herein or illustrated in the drawings hereof. The subject matter described herein is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions, types of materials and physical characteristics described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. § 112(f), unless and until such claim limitations expressly use the phrase “means for” followed by a statement of function void of further structure.

What is claimed is:

1. A system, comprising:
a lead configured to be located within a septal wall;
a monitor configured to obtain cardiac activity signals;
memory configured to store program instructions;
one or more processors that, when executing the program instructions, are configured to:
obtain morphology data related to the cardiac activity signals indicative of the lead located at different depths within the septal wall, the morphology data including a set of data values associated with different depths of the lead within the septal wall; and
determine when the lead is located at a target depth within the septal wall based on the morphology data.

2. The system of claim 1, wherein the target depth locates the lead proximate to the left bundle branch (LBB).

3. The system of claim 1, wherein the morphology data includes QRS width of consecutive heartbeats, and the one or more processors are configured to determine that the lead is located proximate the LBB based on a change in the QRS width from a first QRS width to a second QRS width.

4. The system of claim 3, wherein the change in the QRS width is greater than a QRS width threshold change between the first QRS width and the second QRS width.

5. The system of claim 4, wherein the first QRS width is obtained from a first heartbeat and the second QRS width is obtained from a second heartbeat, and the first heartbeat and second heartbeat are not consecutive heartbeats.

6. The system of claim 1, wherein the morphology data includes a peak-to-peak amplitude of consecutive heartbeats, and the one or more processors are configured to determine that the lead is located proximate the LBB based on a change in a peak-to-peak amplitude value between a first peak-to-peak amplitude and a second peak-to-peak amplitude.

7. The system of claim 6, wherein the change in peak-to-peak amplitude is greater than a threshold change between the first peak-to-peak amplitude and the second peak-to-peak amplitude.

8. The system of claim 1, wherein the determine operation includes determining when the data values satisfy a criteria of interest.

9. The system of claim 8, wherein the determine operation further comprises comparing the data values, within the set of data values, to one another to determine a relation; and determining when the relation satisfies the criteria of interest.

10. The system of claim 9, wherein the criteria of interest is at least one of change in QRS width, or a change in a peak-to-peak amplitude.

11. The system of claim 1, wherein the target depth includes a point where the lead is proximate the LBB and does not perforate a LV septum side wall distal wall of the septal wall.

12. The system of claim 1, wherein the lead is a first electrode of a leadless IMD.

13. A computer implemented method, wherein under control of one or more processors configured with specific executable instructions, the method comprises:
obtaining morphology data indicative of a morphology of consecutive beats of a heart including a set of data values associated with different depths of a lead within the septal wall; and
determining when the lead is located at a target depth within the septal wall based on the morphology data.

14. The method of claim 13 further comprising:
determining that the lead is located proximate the LBB when the morphology data indicates a change in QRS width is above a QRS width change threshold.

15. The method of claim 13, further comprising:
determining that the lead is located proximate the LBB when the morphology data indicates a change in peak-to-peak amplitude is above a peak-to-peak amplitude change threshold.

16. The method of claim 13, wherein the determining operation includes determining when the data values satisfy a criteria of interest.

17. The method of claim 16, wherein the determining operation further comprises comparing the data values, within the set of data values, to one another to determine a relation; and determining when the relation satisfies the criteria of interest.

18. The method of claim 17, wherein the criteria of interest is at least one of change in QRS width, or a change in a peak-to-peak amplitude.

19. The method of claim 13, wherein the target depth includes a point where the lead is proximate the LBB and does not perforate a LV septum side wall distal wall of the septal wall.

20. The method of claim 13, wherein obtaining morphology data indicative of the morphology of the consecutive beats of the heart comprises monitoring, with a monitor, the consecutive beats of the heart as the lead is inserted into the septal wall.
